(19) United States
(12) Patent Application Publication
(10) Pub. No.: US 2024/0350250 A1
(43) Pub. Date: Oct. 24, 2024

(54) Endograft Device For Treating Ruptures In A Blood Vessel

(71) Applicant: Universitat Heidelberg, Heidelberg (DE)

(72) Inventor: Matthias Karck, Heidelberg (DE)

(73) Assignee: Universitat Heidelberg, Heidelberg (DE)

(21) Appl. No.: 18/682,257

(22) PCT Filed: Aug. 8, 2022
(86) PCT No.: PCT/EP2022/072181

§ 371 Date: Feb. 8, 2024

(30) Foreign Application Priority Data
Aug. 9, 2021 (EP) 21190269.7

(51) Int. Cl. A61F 2/07; A61F 2/966

(52) U.S. Cl. CPC A61F 2/07; A61F 2/966; A61F 2002/077; A61F 2002/9665; A61F 2210/0004; A61F 2210/0009; A61F 2210/0014; A61F 2210/0057; A61F 2210/0066; A61F 2210/0071; A61F 2220/0008; A61F 2220/0075; A61F 2230/0069; A61F 2250/0039

(57) ABSTRACT

The present invention relates to an endograft device (101) for treatment of ruptures (104.9) in one or more inner layers of a blood vessel (104), in particular, an aorta, comprising an anchoring unit (101.1) and a sleeve unit (101.2). The endograft device (101) defines an upstream endograft end (101.3) and a downstream endograft end (101.4), upstream and downstream being defined, in an implanted state of the endograft device (101), in relation to a general natural blood flow defining a blood flow direction within the blood vessel (104). The anchoring unit (101.1) is a collapsible unit located at the upstream endograft end (101.3), wherein the anchoring unit (101.1) is configured to anchor, in the implanted state, the endograft device (101) within the blood vessel (104) by engaging an inner surface of the blood vessel (104) with an expanded anchoring section (101.5). The sleeve unit (101.2) is located downstream of the anchoring unit (101.1), wherein the sleeve unit (101.2) is a thin-walled foldable element defining a longitudinal direction, a radial direction, a circumferential direction, an upstream sleeve unit end (101.7) and a upstream sleeve unit end (101.8). The sleeve unit (101.2) comprises an unsupported section (101.9) located at the upstream sleeve unit end (101.7), wherein the unsupported section (101.9) extends along the longitudinal direction and, at least in the implanted state, is unsupported over its entire circumference. The sleeve unit (101.2) is configured to be expanded, in the implanted state, to rest against an inner wall of the blood vessel (104) to sealingly cover the rupture (104.9) with the unsupported section (101.9).

### BACKGROUND OF THE INVENTION

The present invention relates to an endograft device for treatment of ruptures in one or more inner layers of a blood vessel, in particular, an aorta, comprising an anchoring unit and a sleeve unit. The endograft device defines an upstream endograft end and a downstream endograft end, upstream and downstream being defined, in an implanted state of the endograft device, in relation to a general natural blood flow defining a blood flow direction within the blood vessel. The anchoring unit is a collapsible unit located at the upstream endograft end, wherein the anchoring unit is configured to anchor, in the implanted state, the endograft device within the blood vessel by engaging an inner surface of the blood vessel with an expanded anchoring section. The sleeve unit is located downstream of the anchoring unit, wherein the sleeve unit is a thin-walled foldable element defining a longitudinal direction, a radial direction, a circumferential direction, an upstream sleeve unit end and a downstream sleeve unit end. The invention further relates to a corresponding kit comprising the endograft device and a catheter device for implanting the endograft device. It further relates to a catheter device for implanting the endograft device.

The main human artery, the aorta, consists of three layers, the inner layer (intima), the middle layer (media) and the outer layer (adventitia). When, for example, as the result of a traumatic event or spontaneously, a rupture in the intima and the media occurs, a life-threatening condition arises in that a so called aneurysm is formed in a process called “acute aortic dissection”. More precisely, blood flows through the rupture from the aorta into a gap typically forming between the media and the adventitia. As the adventitia is a relatively thin, yet quite elastic layer a so called aneurysmal sac is formed. This aneurysmal sac gradually fills with blood and has an increasing risk of rupture and, hence, of formation of a life-threatening leak in the aorta.

Treatment of this condition usually consists of internally sealingly bridging the rupture and the aneurysm with an endograft device (often referred to as an aortal endograft or aortic stent) as it is known, for example, from US 2004/0098096 A1 (Eton; the entire disclosure of which is incorporated herein by reference) and EP 1 916 965 B1 (Goldmann et al.; the entire disclosure of which is incorporated herein by reference). These known endograft devices are typically implanted using a suitable catheter device inserted into the blood vessel and advanced to the ruptured vessel section. The catheter device typically holds the endograft device in a collapsed state and then releases the endograft device at the ruptured vessel section.

Such endograft devices for treating acute aortic dissection typically rely on a sealing sleeve unit which is expanded and internally supported at its respective upstream and downstream ends by anchoring units, such as expandable stent units. The expanded anchoring units firmly press the sealing sleeve unit against the blood vessel wall (upstream and downstream of the rupture) to bypass the aneurysm and prevent leakage of blood into the aneurysmal sac. If this bridging procedure is successful, no more blood can enter the aneurysmal sac and the risk of a life-threatening endoleak is banned. Moreover, after successful sealing of the rupture, the blood in the aneurysmal sac forms a stable blood clot sealing the rupture. In this case, the stability of the aorta is largely restored.

A problem arising with these procedures is that acute aortic dissection often happens close to locations where large blood vessels branch off the aorta, such as, for example, at the aortic arch where the arteria subclavia branches off. Here, due to the location of the rupture close to or in the rounded vessel juncture (which obviously has to be kept open), such conventional endograft devices may not always be able to completely close the rupture. Such an incomplete closure of the rupture leads to a so-called “type I endoleak” where blood still flows past the endograft device into the rupture and thus into the aneurysmal sac. As a result, treatment fails and the risk of a life-threatening endoleak still exists.

### SUMMARY OF THE INVENTION

Thus, it is the object of the present invention to provide an endograft device and a catheter device for applying such an endograft which do not show the disadvantages described above, or at least show them to a lesser extent, and, in particular, allow in a simple, and efficient manner improved treatment of ruptures in blood vessels at locations with complex vessel geometry.

The above objects are achieved by an endograft device according claim 1 as well as by a catheter device according to claim 13.

The present invention is based on the technical teaching that, it is possible to achieve, in a simple and efficient manner, improved treatment of ruptures in blood vessels at locations with complex vessel geometry if the anchoring unit, in the implanted state of the endograft device, is located upstream of the thin and flexible sleeve unit such that at least the upstream part of the sleeve unit sealingly covering the rupture is unsupported along its circumference. This longitudinally non-overlapping arrangement of the flexible sleeve unit and the anchoring unit frees considerable adaptation capacity of the expanding sleeve unit, especially in the radial direction of the endograft device or blood vessel, respectively. This is due to the fact that constraints in the sleeve unit's freedom to adapt to the vessel topography which are typically imposed by a (longitudinally) overlapping or embedded anchoring unit are largely removed. This allows the unsupported sleeve unit to at least largely freely and closely conform (under the supporting influence of the blood pressure inside the blood vessel) to the vessel topography around the rupture, and, therefore, tightly and reliably seal the rupture.

As clotting blood in the sealed rupture and the possibly already formed aneurysmal sac comparatively quickly restores vessel stability, the thin walled sealing unit can be made of a bioresorbable material which quickly leads to restoration of the natural flow conditions in the vessel.

Hence, according to one aspect, the present invention relates to an endograft device for treatment of ruptures in one or more inner layers of a blood vessel, in particular, an aorta, comprising an anchoring unit and a sleeve unit. The endograft device defines an upstream endograft end and a downstream endograft end, upstream and downstream being defined, in an implanted state of the endograft device, in relation to a general natural blood flow defining a blood flow direction within the blood vessel. The anchoring unit is a collapsible unit located at the upstream endograft end, wherein the anchoring unit is configured to anchor, in the implanted state, the endograft device within the blood vessel by engaging an inner surface of the blood vessel with an expanded anchoring section. The sleeve unit is located downstream of the anchoring unit, wherein the sleeve unit is a thin-walled foldable element defining a longitudinal direction, a radial direction, a circumferential direction, an upstream sleeve unit end and a downstream sleeve unit end. The sleeve unit comprises an unsupported section located at the upstream sleeve unit end, wherein the unsupported section extends along the longitudinal direction and, at least in the implanted state, is unsupported over its entire circumference. The sleeve unit is configured to be expanded, in the implanted state, to rest against an inner wall of the blood vessel to sealingly cover the rupture with the unsupported section.

It will be appreciated that the unsupported part of the sleeve unit may have any desired length suitable to provide sufficient adaptability, especially in the radial direction, of the thin walled sleeve unit to the vessel topography. With certain preferred variants, the unsupported section, along the longitudinal direction, extends over at least 10%, preferably at least 20%, more preferably 25% to 100%, in particular, 40% to 80%, of the sleeve unit. With certain preferred variants, the unsupported section, along the longitudinal direction, extends over mm to 100 mm, preferably 20 mm to 80 mm, more preferably 40 mm to 60 mm. In any of these cases, particularly suitable configurations with high adaptability of the expanding sleeve unit to complex vessel topographies may be achieved.

It will be appreciated that the sleeve unit may be unsupported along its entire length. With certain variants, however, the sleeve unit may comprise at least one supported section, wherein the at least one supported section is located adjacent to the unsupported section and at a distance from the upstream sleeve unit end. The at least one supported section, in the implanted state, is supported over its circumference, by at least one expandable support unit. The at least one supported section may be located at any desired location suitably spaced from the upstream sleeve unit end. In particular, it may be located directly at the downstream sleeve unit end. The latter has the advantage that a sealing contact with the blood vessel at the downstream end (typically interfacing with a less complex vessel topography of typically generally circular cross section), may reliably prevent inflow of blood into the interface between the sleeve unit and the blood vessel, thereby ensuring close and sealing contact between the sleeve unit and the blood vessel.

It will be appreciated that, generally, any desired number of supported sections and further unsupported sections may be provided in any desired sequence. Moreover, one or more supported sections may extend over any desired and suitable fraction of the sleeve unit. Preferably, the at least one supported section, along the longitudinal direction, extends over at least 10%, preferably at least 15%, more preferably 20% to 60%, in particular, 30% to 50%, of the sleeve unit. With certain variants, the at least one supported section, along the longitudinal direction, extends over 10 mm to 60 mm, preferably 15 mm to 50 mm, more preferably 20 mm to 40 mm. In any of these cases, beneficial sealing properties may be achieved.

Support of the sleeve unit in the at least one supported section may be achieved in any desired and suitable wax. For example, the support unit may be embedded, at least in part, in the sleeve unit. With certain variants, the at least one supported section, in the radial direction, is supported by the at least one support unit on a (radially) outer side and/or on an (radially) inner side of the sleeve unit.

It will be appreciated that the anchoring unit and the sleeve unit may be configured and mutually adapted in any desired and suitable way which yields sufficient freedom to the sleeve unit to snugly conform to the blood vessel topography, preferably in an essentially stress-free manner (in particular, in the circumferential direction of the endograft device).

With certain variants, the anchoring section may be configured to be expanded, in the implanted state, to a maximum anchoring unit diameter, a circle having the maximum anchoring unit diameter defining a reference circumferential length of the anchoring unit. The sleeve unit, in a relaxed and fully expanded state free of stress in the circumferential direction, at the upstream sleeve unit end, has a relaxed expanded circumferential length. Here, the anchoring unit and the sleeve unit are arranged and configured such that, in the implanted state, the relaxed expanded circumferential length of the upstream sleeve unit end is larger than the reference circumferential length of the anchoring unit. The enlarged circumferential length of the relaxed expanded sleeve unit over the reference circumferential length of the anchoring unit, provides a beneficial topography adaptation reserve to the sleeve unit, especially at the upstream-most part or the upstream boundary, respectively, of the sleeve unit.

In these cases, for example, the (accordingly enlarged or “oversized”) upstream sleeve unit end may easily conform to the possibly complex topography of the blood vessel in the area of a rupture to properly cover and seal the rupture. This may happen in a “relaxed” manner, i.e., substantially free from circumferential resetting stresses, which is beneficial in that the lack of such circumferential resetting stresses ensures that the sealing contact between the sleeve unit and the blood vessel can be maintained at any time over the required treatment period. Such a relaxed adaptation or conformation, respectively, to the vessel topography may be achieved, for example, by local folding or plying of the thin-walled and highly flexible sleeve unit.

It will be appreciated that any desired and suitable oversize of the upstream sleeve unit end over the anchoring unit may be chosen. With certain beneficial variants, the anchoring unit and the sleeve unit are arranged and configured such that, in the implanted state, the relaxed expanded circumferential length of the upstream sleeve unit end is at least 101%, preferably at least 105%, more preferably 110% to 130%, in particular, 115% to 125%, of the reference circumferential length of the anchoring unit. With certain variants, the relaxed expanded circumferential length of the upstream sleeve unit end is 55 mm to 75 mm, preferably 60 mm to 70 mm, more preferably 62 mm to 65 mm. With certain particularly well suited variants, the reference circumferential length of the anchoring unit is 50 mm to 70 mm, preferably 55 mm to 65 mm, more preferably 58 mm to 62 mm. In any of these cases, particularly beneficial configurations with properly large topography adaptation reserve of the sleeve unit may be achieved.

It will be appreciated that the sleeve unit may have any desired and suitable tubular shape which can be adapted to the specific topography of the blood vessel at the target location (i.e., the ruptured location). For example, the sleeve may be adapted to the course of the central axis of the blood vessel at the target location, e.g., to the curvature of the aorta in the region of the aortic arch. In particularly simple configurations, the sleeve unit, in a relaxed (i.e., essentially stress free) and fully expanded state, may be an essentially straight component with a straight central longitudinal axis.

With certain variants, the sleeve unit is configured to have, in a relaxed and fully expanded state free of stress in the circumferential direction, a shape that is at least one of (i) at least section-wise at least substantially cylindrical and (ii) flared, along the longitudinal direction, towards at least one of the upstream sleeve unit end and the downstream sleeve unit end. Hence, the sleeve unit may have one or more essentially cylindrical (typically with circular cross section) sections. In addition or as an alternative, one or both end sections may flare towards the respective end of the sleeve unit.

With certain variants, the sleeve unit, in the relaxed and fully expanded state, along the longitudinal direction, has a sleeve unit minimum diameter and is flared towards the upstream sleeve unit end to have an upstream flared diameter at the upstream sleeve unit end. The upstream flared diameter may be at least one of (i) 101%, preferably at least 103%, more preferably 105% to 130%, in particular, 110% to 125%, of the sleeve unit minimum diameter, and (ii) at least 18 mm to 25 mm, preferably 19 mm to 23 mm, more preferably 20 mm to 22 mm. In either case particularly beneficial configurations with proper sealing contact of the upstream sleeve unit end with the blood vessel may be achieved even in situations with complex vessel topography in the surroundings of the rupture.

With certain variants, the sleeve unit, in the relaxed and fully expanded state, along the longitudinal direction, has a sleeve unit minimum diameter and is flared towards the downstream sleeve unit end to have a downstream flared diameter at the downstream sleeve unit end, the downstream flared diameter being at least one of (i) 101%, preferably at least 103%, more preferably 105% to 130%, in particular, 110% to 125%, of the sleeve unit minimum diameter, and (ii) at least 18 mm to 25 mm, preferably 19 mm to 23 mm, more preferably 20 mm to 22 mm. In either case particularly beneficial configurations with proper sealing contact of the downstream sleeve unit end with the blood vessel may be achieved. The flared configuration at the downstream end may be particularly beneficial in cases where also the downstream end of the sleeve unit is unsupported.

With certain variants, the anchoring section, in the implanted state, has a maximum anchoring unit diameter and the sleeve unit, in the relaxed and fully expanded state, along the longitudinal direction, has a sleeve unit minimum diameter, the sleeve unit minimum diameter being at least one of (i) 101%, preferably at least 103%, more preferably 105% to 130%, in particular, 110% to 125%, of the maximum anchoring unit diameter, and (ii) at least 16 mm to 22 mm, preferably 17 mm to 21 mm, more preferably 18 mm to 20 mm. By this means, particularly suitable and beneficial oversized configurations of the sleeve unit (over the anchoring unit) may be achieved.

It will be appreciated that the flared configuration may basically be achieved in any desired and suitable way. With certain variants, the sleeve unit, to provide the flared shape in the relaxed and fully expanded state, has a collapsed state where at least one folded section is formed within the sleeve unit. Arbitrary suitable folding or plying schemes may be used. Preferably, a large number of folds (e.g., at least 10 to 20 folds at the circumference) is used at the latter also promotes flexible and close adaptation to complex vessel topographies. Furthermore, preferably, the folds extend at least predominantly along the longitudinal direction to ensure simple and proper unfolding upon expansion from the collapsed state. For example, a folding scheme in the manner of a skirt may be beneficially used.

It will be appreciated that the sleeve unit may be located immediately adjacent to the anchoring unit. With certain variants, however, the sleeve unit is spaced, in the longitudinal direction, from the anchoring unit. Such a spacing may beneficially provide further freedom of adaptation to the sleeve unit. Generally, an increased spacing or distance yields increased freedom of adaptation.

With certain variants, the anchoring section is configured to be expanded, in the implanted state, to a maximum anchoring unit diameter, and the sleeve unit, in the longitudinal direction, is located at a distance D from the anchoring unit, the distance D being at least 5%, preferably at least 10%, more preferably 15% to 100%, in particular, 25% to 75%, of the maximum anchoring unit diameter. With certain variants, the sleeve unit, in the longitudinal direction, is located at a distance D from the anchoring unit, the distance D being at least 1 mm to 20 mm, preferably 3 mm to 15 mm, more preferably 5 mm to 10 mm. In any of these cases, particularly suitable configurations with good freedom of adaptation (to even highly complex vessel topographies) are achieved.

It will be appreciated that the sleeve unit may be (longitudinally) linked to the anchoring unit in any desired and suitably way which preferably supports adaptation of the sleeve unit to the vessel topography. With certain variants, the sleeve unit is linked, in the longitudinal direction, to the anchoring unit by linking means of the endograft device, in particular, by a linking section of the endograft device. With certain variants, the linking means comprises a plurality of linking elements mutually spaced and distributed, in particular, evenly distributed, along the circumferential direction. With certain variants, the linking means comprises a plurality of linking elements, at least one of the linking elements being a slender element elongated along the longitudinal direction. With certain variants, the linking means comprises a plurality of linking elements, at least one of the linking elements being an undulated element undulated along the longitudinal direction. With certain variants, the linking means comprises a plurality of linking elements, at least one of the linking elements being a flexible element exhibiting a high flexibility in a direction transverse to the longitudinal direction. Either of these configurations is particularly beneficial in that the design and flexibility of the linking elements supports adaptation of the sleeve unit to the vessel topography.

With certain variants, the linking means comprises a plurality of linking elements, at least one of the linking elements being one of a filament element, a surgical suture filament, a tongue element protruding from said sleeve unit, and a wire element. Either of these configurations is particularly beneficial in that the design and flexibility of the linking elements supports adaptation of the sleeve unit to the vessel topography.

With certain variants, the linking means comprises a perforated linking sleeve section, the linking sleeve section having a degree of perforation (i.e., a ratio between the open surface allowing through-flow of blood and the overall surface) which is at least 85%, preferably at least 90%, more preferably 86% to 94%, in particular, 89% to 92%, the perforated linking sleeve section, in particular, being formed monolithically with at least one of the sleeve unit and the anchoring section. Here, a particularly simple to manufacture and robust configuration may be achieved.

It will be appreciated that the sleeve unit may have any desired and suitable configuration yielding a thin-walled and highly flexible component which snugly conforms to the vessel topography. Preferably, the sleeve unit has a wall thickness ranging from 0.05 mm to 2.0 mm, preferably 0.1 mm to 1.0 mm, more preferably 0.25 mm to 0.5 mm. Any desired and suitable materials may be used for the sleeve unit. With certain variants, the sleeve unit is made from a material selected from a material group consisting of a polymer material, polyethylene (PE), polytetrafluoroethylene (PTFE), polyurethane (PU), a bioresorbable material, a lactide caprolactone, allogeneic pericardium, matrix based and/or tissue engineered material, and combinations thereof.

It will be appreciated that the anchoring and support functionality as described herein may be achieved in any desired and suitable way ensuring proper contact forces at the blood vessel. With certain variants, to this end, the anchoring unit and/or a support unit supporting the sleeve unit at the downstream sleeve unit end comprises a collapsible and expandable structure, in particular, a self-expanding structure. With certain preferred variants, the collapsible and expandable structure is made from a material selected from a material group consisting of a shape memory material, a metal, a nickel titanium alloy, a polymer material, a bioresorbable material, a magnesium based material, and combinations thereof. With certain variants, the collapsible and expandable structure comprises ate least one of a grid and a wire structure. With certain variants, the collapsible and expandable structure comprises at least one stent element. Any of these variants yields particularly beneficial designs with proper and stable application of contact forces.

The present invention further relates to a kit for treatment of ruptures in one or more inner layers of a blood vessel, in particular, an aorta, comprising at least one endograft device according to the invention and a catheter device. The catheter device is preferably configured for percutaneous insertion into a blood vessel of a patient. The catheter device has a proximal end and a distal end, wherein the catheter device is configured to receive, in a catheter insertion state, the endograft device within an endograft receptacle formed at the distal end of the catheter device, the endograft device being in a collapsed state when received in the endograft receptacle. The catheter device is further configured to release, in an endograft release state, the endograft device from the endograft receptacle. With such a kit the above variants and advantages can be achieved to the same extent, such that reference is made to the explanations given above.

Preferably, the catheter device comprises an inner catheter core and an outer catheter sleeve, wherein the endograft receptacle is formed between the inner catheter core and the outer catheter sleeve when the outer catheter sleeve is in a distally advanced state. The outer catheter sleeve may be proximally retractable with respect to the inner catheter core to release the endograft device. With certain variants, the anchoring unit and/or a support unit of the endograft device supporting the sleeve unit at the downstream sleeve unit end may comprise a self-expanding structure expanding upon release from the outer catheter sleeve. By this means, particularly simple release may be achieved. With certain variants, the inner catheter core may be configured to at least support radial expansion of the sleeve unit, in particular, when retracting the inner catheter core, in the endograft release state, proximally from the endograft device. By this means, particularly good adaptation and snug fit of the sleeve unit to the blood vessel topography may be achieved.

The present invention further relates to a catheter device for inserting an endograft device according to the invention into a blood vessel, in particular, an aorta, for treatment of ruptures in one or more inner layers of the blood vessel. The catheter device comprises an inner catheter core and an outer catheter sleeve. An endograft receptacle is formed between the inner catheter core and the outer catheter sleeve for receiving the endograft device when the outer catheter sleeve is in a distally advanced state. The outer catheter sleeve is proximally retractable with respect to the inner catheter core to release the endograft device from the endograft receptacle in an endograft release state. The inner catheter core is configured to at least support radial expansion of the sleeve unit, in particular, when retracting the inner catheter core, in the endograft release state, proximally from the endograft device. By this means, particularly good adaptation and snug fit of the sleeve unit to the blood vessel topography may be achieved. With such a catheter device the above variants and advantages can be achieved to the same extent, such that reference is made to the explanations given above.

Support of the radial expansion of the sleeve unit may be achieved in any desired and suitable way. With preferred variants, the inner catheter core comprises a catheter core expansion section which is configured to radially expand to at least support radial expansion of the sleeve unit. With certain variants the inner catheter core comprises a catheter core expansion section which is configured to radially expand when the outer catheter sleeve is proximally retracted with respect to the inner catheter core. In either case, simple and proper support of the radial expansion of the sleeve unit may be achieved.

Expansion of the catheter core expansion section may be achieved in any suitable way. For example, the catheter core expansion section may be made from an expandable material forming one or more suitable chambers to be filled with a suitable fluid to provide expansion. With particularly simple yet robust and efficient variants, the inner catheter core comprises a plurality of elastic arms, the elastic arms being configured to radially expand and engage the sleeve unit to support radial expansion of the sleeve unit when the outer catheter sleeve is proximally retracted with respect to the inner catheter core. This configuration has the great advantage that the elastic arms may be configured to only marginally block blood flow within the blood vessel which further supports expansion of the sleeve unit.

With certain variants, the elastic arms are distributed, in particular, at least substantially evenly distributed, along a circumference of the inner catheter core. With certain variants, the elastic arms are configured to be retracted proximally with respect to the sleeve unit and to slide along the sleeve unit when being retracted proximally. This achieves a particularly simple configuration with proper contact between the sleeve unit and the blood vessel along the length of the sleeve unit. With certain variants, the elastic arms are configured be retracted into the outer catheter sleeve after having been proximally retracted from the sleeve unit. This enables simple and safe removal of the catheter device after release of the endograft device. Preferably, the inner catheter core and the outer catheter sleeve are configured such that a proximal retraction motion of the elastic arms and a proximal retraction motion of the outer catheter sleeve are synchronized such that the proximal retraction motion of the elastic arms follows the proximal retraction motion of the outer catheter sleeve with a predefined delay. This also enables simple and safe release of the endograft device.

It will be appreciated that, with certain preferred variants, radio-opaque markers are integrated at certain suitable locations, e.g., at the upstream and/or downstream ends of both, the anchoring unit and sleeve unit and at various additional segments of the entire endograft device in order to enable quick and simple proper placement of the endograft device.

Further aspects and embodiments of the invention become apparent from the dependent claims and the following description of preferred embodiments, which refers to the attached figures. All combinations of the disclosed features, regardless of whether they are the subject of a claim or not, are within the scope of protection of the invention.

### BRIEF DESCRIPTION OF THE DRAWINGS

FIG. 1 is a schematic view of a preferred embodiment of an endograft device according to the present invention in a state implanted in the region of an aortic arch of a patient;

FIG. 2 is a schematic sectional view of the endograft device of FIG. 1 in a relaxed, fully expanded and non-implanted state.

FIG. 3 is a schematic sectional view of part of a preferred embodiment of a catheter device according to the present invention receiving the endograft device of FIG. 2 in a collapsed state.

FIG. 4 is a schematic sectional view (similar to FIG. 3) of the catheter device of FIG. 3 in a state during release of the endograft device.

### DETAILED DESCRIPTION OF THE INVENTION

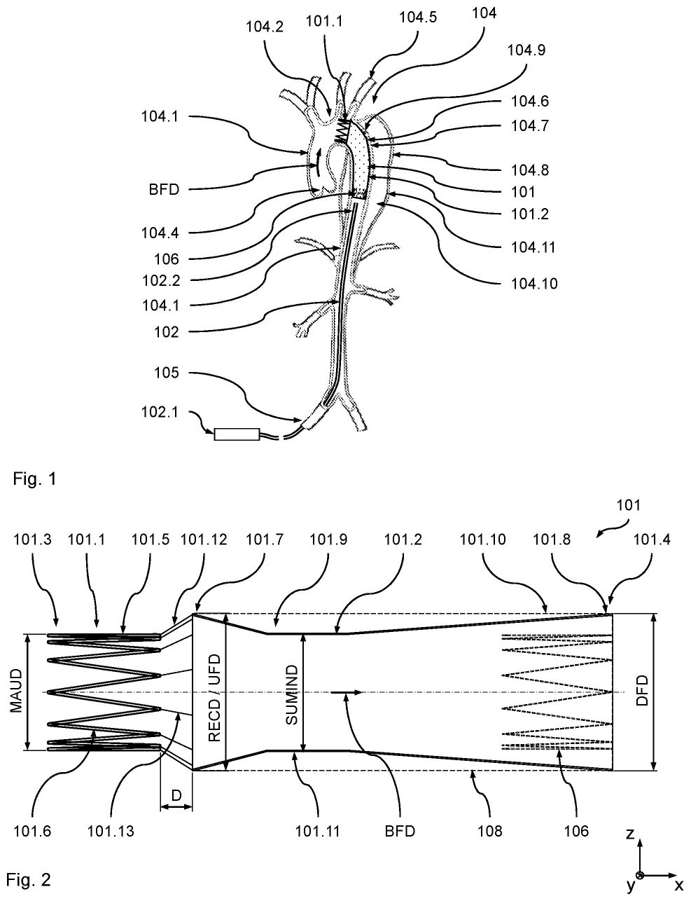

With reference to FIGS. 1 to 4 preferred embodiments of an endograft device 101 according to the present invention and a preferred embodiment of a catheter device 102 according to the present invention, both forming part of a preferred embodiment of a kit 103 according to the present invention will now be described in greater detail.

In order to simplify the explanations given below, an xyz-coordinate system has been introduced into the Figures, wherein the x-axis designates the longitudinal axis (or direction, respectively) of the endograft device 101, while the y-axis and the z-axis both designate the radial directions of the endograft device 101 (the same, of course, applies for the catheter device 102).

FIG. 1 shows parts of the human vascular system, in particular, the aorta 104, which includes the ascending aorta 104.1, the aortic arch 104.2 and the descending aorta 104.3. Oxygenated blood is fed to from the left ventricle of the heart (not shown) through the heart valve 104.4 to generate a general natural blood flow into the ascending aorta 104.1, through the aortic arch 104.2 and into the descending aorta from where it is distributed with the human body. For example, the left subclavian artery (or subclavia sinistra) 104.5 branches off from the aortic arch 104.2 to feed, among others, the vascular system of the left arm.

The aorta 104 consists of three layers, the inner layer (intima) 104.6, the middle layer (media) 104.7 and the outer layer (adventitia) 104.8. When, for example, as the result of a traumatic event, a rupture 104.9 in the intima 104.6 and the media 104.7 occurs, a life-threatening condition arises in that a so called aneurysm is formed in a process called “acute aortic dissection”. More precisely, blood flows through the rupture 104.9 from the aorta 104 into a gap 104.10 typically forming between the media 104.7 and the adventitia 104.9. As the adventitia 104.8 is a relatively thin, yet quite elastic layer a so called aneurysmal sac 104.11 is formed. This aneurysmal sac 104.11 gradually fills with blood and has an increasing risk of rupture and, hence, formation of a life-threatening endoleak in the aorta 104.

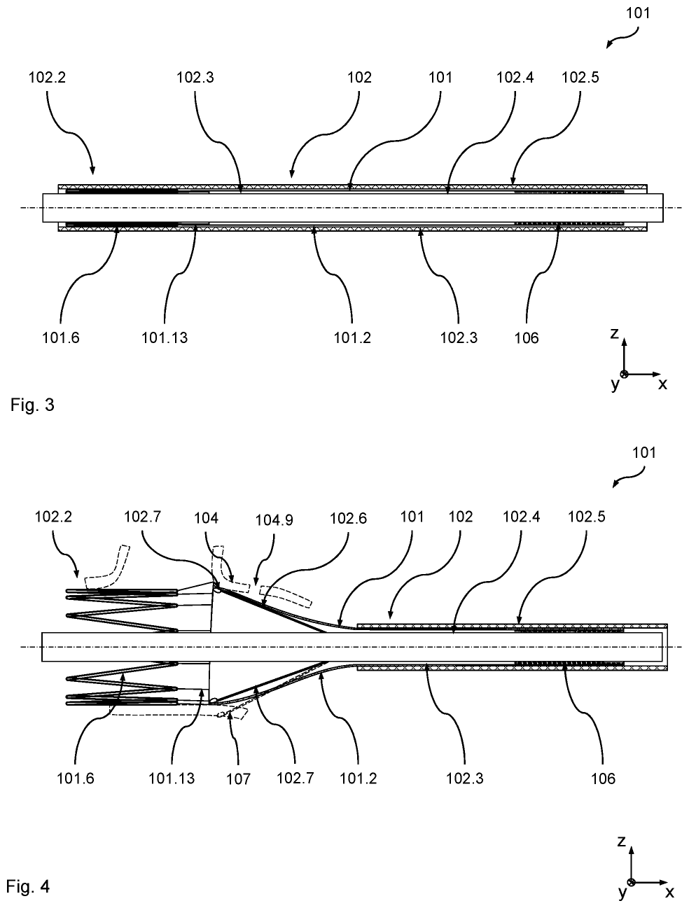

Treatment of this life-threatening condition is provided by internally sealingly covering the rupture 104.9 and stopping formation and further growth of aneurysm using the endograft device 101. The endograft device 101 is implanted using the catheter device 102 (see FIGS. 3 and 4) inserted into the aorta 104 via a percutaneous access (not shown) at the femoral artery (arteria femoralis) 105 and advanced to the ruptured section of the aorta 104. The catheter device 102 holds the endograft device 101 in a collapsed state and then releases the endograft device 101 at the ruptured section (as will be explained in greater detail below). The catheter device 102 is then again withdrawn from the patient. After this sealing procedure is completed, no more blood can enter the aneurysmal sac 104.11 and the risk of a life-threatening endoleak is banned. Moreover, the blood in the aneurysmal sac 104.11 forms a stable blood clot permanently sealing the rupture 104.9. In this case, the stability of the aorta 104 is largely restored.

As can be seen from FIGS. 1 and 2, the endograft device 101 comprises an anchoring unit 101.1 and a sleeve unit 101.2. The endograft device 101 defines an upstream endograft end 101.3 and a downstream endograft end 101.4, upstream and downstream being defined, in the implanted state of the endograft device 101 (see FIG. 1), in relation to the general natural blood flow defining a blood flow direction BFD within the blood vessel, here the aorta 104.

The anchoring unit 101.1 is a collapsible and expandable unit located at the upstream endograft end 101.3. In the implanted state (see FIG. 1), the anchoring unit 101.1 anchors the endograft device 102 within the aorta 104 by engaging an inner surface of the aorta 104 with an expanded anchoring section 101.5 formed by a stent element 101.6.

The sleeve unit 101.2 is located downstream of the anchoring unit 101.1. The sleeve unit 101.2 is a thin-walled foldable element defining a longitudinal direction LD (parallel to the x axis in FIG. 2), a radial direction RD (e.g. parallel to the z axis in FIG. 2), and a circumferential direction CD. The sleeve unit 101.2 has an upstream sleeve unit end 101.7 and a downstream sleeve unit end 101.8. The sleeve unit 101.2 comprises an unsupported section 101.9 located at the upstream sleeve unit end 101.7. The unsupported section 101.9 extends along the longitudinal direction LD and, in the implanted state (see FIG. 1), is unsupported over its entire circumference. It will be appreciated that unsupported means that there is no further supporting structure or the like which supports the sleeve unit 101.2 in the radial direction RD. In the implanted state of FIG. 1, the sleeve unit 101.2 is expanded to rest against an inner wall of the aorta 104 to sealingly cover the rupture 104.9 with the unsupported section 101.9.

It will be appreciated that the unsupported part 101.9 of the sleeve unit 101.2 may have any desired length suitable to provide sufficient adaptability, especially in the radial direction RD, of the thin walled sleeve unit 101.2 to the vessel topography. With certain preferred variants, the unsupported section 101.9, along the longitudinal direction LD, extends over at least 10%, preferably at least 20%, more preferably 25% to 100%, in particular, 40% to 80%, of the sleeve unit 101.2. With certain preferred variants, the unsupported section 101.9, along the longitudinal direction LD, extends over 10 mm to 100 mm, preferably 20 mm to 80 mm, more preferably 40 mm to 60 mm. In any of these cases, particularly suitable configurations with high adaptability of the expanding sleeve unit 101.2 to complex vessel topographies may be achieved.

It will be appreciated that the sleeve unit 101.2 may be unsupported along its entire length. With certain variants, however, the sleeve unit may comprise at least one supported section 101.10 located adjacent to the unsupported section 101.9 and at a distance from the upstream sleeve unit end 101.7. In the implanted state of FIG. 1, the supported section 101.10, may be supported over its circumference by at least one expandable support unit, e.g., a collapsible and expandable stent unit, as it is indicated in FIGS. 1 and 2 by the dashed contour 106.

The supported section 101.10 may be located at any desired location suitably spaced from the upstream sleeve unit end 101.7. In particular, it may be located directly at the downstream sleeve unit end 101.8. The latter has the advantage that a sealing contact with the aorta 104 at the downstream end 101.8 (typically interfacing with a less complex vessel topography of typically generally circular cross section), may reliably prevent inflow of blood into the interface between the sleeve unit 101.2 and the aorta 104, thereby ensuring close and sealing contact between the sleeve unit 101.2 and the aorta 104.

It will be appreciated that, generally, any desired number of supported sections 101.10 and further unsupported sections may be provided in any desired sequence. Moreover, one or more supported sections 101.10 may extend over any desired and suitable fraction of the sleeve unit 101.2. Preferably, the supported section 101.10, along the longitudinal direction LD, extends over at least 10%, preferably at least 15%, more preferably 20% to 60%, in particular, 30% to 50%, of the sleeve unit 101.2. With certain variants, the supported section 101.10, along the longitudinal direction LD, extends over 10 mm to 60 mm, preferably 15 mm to 50 mm, more preferably 20 mm to 40 mm. In any of these cases, beneficial sealing properties may be achieved.

Support of the sleeve unit 101.2 in the supported section may be achieved in any desired and suitable wax. For example, the support unit 106 may be embedded, at least in part, in the sleeve unit 101.2. With certain variants, the supported section 101.10, in the radial direction, is supported by the support unit 106 on a (radially) outer side (not shown) and/or on an (radially) inner side of the sleeve unit (see, in particular, FIG. 1).

It will be appreciated that the anchoring unit 101.1 and the sleeve unit 101.2 may be configured and mutually adapted in any desired and suitable way which yields sufficient freedom to the sleeve unit 101.2 to snugly conform to the blood vessel topography of the aorta 104, preferably in an essentially stress-free manner (in particular, in the circumferential direction of the endograft device 102).

In the present example, the anchoring section 101.5 is configured to be expanded, in the implanted state, to a maximum anchoring unit diameter MAUD, wherein a circle having the maximum anchoring unit diameter MAUD then defines a reference circumferential length RCL of the anchoring unit 101.1. The sleeve unit 101.2, in a relaxed and fully expanded state free of stress in the circumferential direction (as shown in FIG. 2), at the upstream sleeve unit end 101.7, has a relaxed expanded circumferential length RECL. In the present example, the relaxed expanded circumferential length RECL corresponds to the circumferential length of a circle having the relaxed expanded circumferential diameter RECD as shown in FIG. 2.

In the present example, the anchoring unit 101.1 and the sleeve unit 101.2 are arranged and configured such that, in the implanted state, the relaxed expanded circumferential length RECL of the upstream sleeve unit end 101.7 is larger than the reference circumferential length RCL of the anchoring unit 101.1. The enlarged circumferential length RECL of the relaxed expanded sleeve unit 101.2 over the reference circumferential length RCL of the anchoring unit 101.1, provides a beneficial topography adaptation reserve to the sleeve unit 101.2, especially at the upstream-most part or the upstream boundary, respectively, of the sleeve unit 101.2.

In these cases, for example, the (accordingly enlarged or “oversized”) upstream sleeve unit end 101.7 may easily conform to the possibly complex topography of the aorta 104 in the area of the rupture 104.9 to properly cover and seal the rupture 109.4. This may happen in a “relaxed” manner, i.e., substantially free from circumferential resetting stresses, which is beneficial in that the lack of such circumferential resetting stresses ensures that the sealing contact between the sleeve unit 101.2 and the aorta 104 can be maintained at any time over the required treatment period. Such a relaxed adaptation or conformation, respectively, to the vessel topography may be achieved, for example, by local folding or plying of the thin-walled and highly flexible sleeve unit 101.2.

It will be appreciated that any desired and suitable oversize of the upstream sleeve unit end 101.7 over the anchoring unit 101.1 may be chosen. With certain beneficial variants, the anchoring unit 101.1 and the sleeve unit 101.2 are arranged and configured such that, in the implanted state, the relaxed expanded circumferential length RECL of the upstream sleeve unit end 101.7 is at least 101%, preferably at least 105%, more preferably 110% to 130%, in particular, 115% to 125%, of the reference circumferential length RCL of the anchoring unit 101.1. With certain variants, the relaxed expanded circumferential length RECL of the upstream sleeve unit end 101.7 is 55 mm to 75 mm, preferably 60 mm to 70 mm, more preferably 62 mm to 65 mm. With certain particularly well suited variants, the reference circumferential length RCL of the anchoring unit 101.1 is 50 mm to 70 mm, preferably 55 mm to 65 mm, more preferably 58 mm to 62 mm. In any of these cases, particularly beneficial configurations with properly large topography adaptation reserve of the sleeve unit 101.2 may be achieved.

It will be appreciated that the sleeve unit 101.2 may have any desired and suitable tubular shape which can be adapted to the specific topography of the aorta 104 at the target location (i.e., the ruptured location). For example, the sleeve unit 101.2 may by adapted to the course of the central axis of the aorta 104 at the target location, e.g., to the curvature of the aorta 104 in the region of the aortic arch 101.2. In particularly simple configurations, as with the present example, the sleeve unit 101.2, in the relaxed (i.e., essentially stress free) and fully expanded state shown in FIG. 2, is an essentially straight component with a straight central longitudinal axis.

In the present example, in the relaxed and fully expanded state free of stress in the circumferential direction, the sleeve unit has a shape that is, in a middle section 101.11, substantially cylindrical and that is flared (along the longitudinal direction) towards both the upstream sleeve unit end 101.7 and the downstream sleeve unit end 101.8.

With certain variants, the sleeve unit 101.2, in the relaxed and fully expanded state of FIG. 2, along the longitudinal direction, has a sleeve unit minimum diameter SUMIND and is flared towards the upstream sleeve unit end 101.7 to have an upstream flared diameter UFD (=RECD) at the upstream sleeve unit end 101.7. The upstream flared diameter UFD may be 101%, preferably at least 103%, more preferably 105% to 130%, in particular, 110% to 125%, of the sleeve unit minimum diameter SUMIND. Preferably, the upstream flared diameter UFD is at least 18 mm to 25 mm, preferably 19 mm to 23 mm, more preferably 20 mm to 22 mm. In either case particularly beneficial configurations with proper sealing contact of the upstream sleeve unit end with the blood vessel may be achieved even in situations with complex vessel topography in the surroundings of the rupture. Furthermore, the sleeve unit 101.2 is flared towards the downstream sleeve unit end 101.8 to have a downstream flared diameter DFD at the downstream sleeve unit end 101.8. The downstream flared diameter DFD may be 101%, preferably at least 103%, more preferably 105% to 130%, in particular, 110% to 125%, of the sleeve unit minimum diameter SUMIND. Preferably, the downstream flared diameter DFD is at least 18 mm to 25 mm, preferably 19 mm to 23 mm, more preferably 20 mm to 22 mm. In either case particularly beneficial configurations with proper sealing contact of the downstream sleeve unit end 101.8 with the aorta 104 may be achieved. As noted, the flared configuration at the downstream end 101.8 may be particularly beneficial in cases where also the downstream end 101.8 of the sleeve unit 101.2 is unsupported.

With certain variants, the sleeve unit minimum diameter SUMIND may be 101%, preferably at least 103%, more preferably 105% to 130%, in particular, 110% to 125%, of the maximum anchoring unit diameter MAUD of the anchoring section. Preferably, the sleeve unit minimum diameter SUMIND is at least 16 mm to 22 mm, preferably 17 mm to 21 mm, more preferably 18 mm to 20 mm. By this means, particularly suitable and beneficial oversized configurations of the sleeve unit 101.2 (over the anchoring unit 101.1) may be achieved.

It will be appreciated that the flared configuration as shown in FIG. 2 may basically be achieved in any desired and suitable way. With certain variants, the sleeve unit 101.2, to provide the flared shape in the relaxed and fully expanded state, has a collapsed state (see FIG. 3) where at least one folded section is formed within the sleeve unit 101.2. Arbitrary suitable folding or plying schemes may be used. Preferably, a large number of folds (e.g., at least 10 to 20 folds at the circumference) is used at the latter also promotes flexible and close adaptation to complex vessel topographies. Furthermore, preferably, the folds extend at least predominantly along the longitudinal direction LD to ensure simple and proper unfolding upon expansion from the collapsed state. For example, a folding scheme in the manner of a skirt may be beneficially used.

It will be appreciated that, with certain variants, the sleeve unit 101.2 may be located immediately adjacent to the anchoring unit 101.1. In the present example, however, the sleeve unit 101.2 is spaced, in the longitudinal direction LD, from the anchoring unit 101.1 by a distance D. Such a spacing may beneficially provide further freedom of adaptation to the sleeve unit 101.2. Generally, an increased spacing or distance D yields increased freedom of adaptation.

In the present example, distance D may be at least 5%, preferably at least 10%, more preferably 15% to 100%, in particular, 25% to 75%, of the maximum anchoring unit diameter MAUD. With certain variants, the distance D may be at least 1 mm to 20 mm, preferably 3 mm to 15 mm, more preferably 5 mm to 10 mm. In any of these cases, particularly suitable configurations with good freedom of adaptation (to even highly complex vessel topographies) are achieved.

It will be appreciated that the sleeve unit 101.2 may be (longitudinally) linked to the anchoring unit 101.1 in any desired and suitable way which preferably supports adaptation of the sleeve unit 101.1 to the vessel topography. In the present example, the sleeve unit 101.2 is linked, in the longitudinal direction, to the anchoring unit 101.1 by linking means 101.12 of the endograft device 101, such as a linking section of the endograft device 101. In the present example, the linking means 101.12 comprises a plurality of linking elements 101.13 mutually spaced and distributed, preferably evenly distributed, along the circumferential direction CD. The linking elements 101.13 are slender elements elongated along the longitudinal direction LD. The linking elements 101.13 are flexible elements exhibiting a high flexibility in a direction transverse to the longitudinal direction LD. With certain variants, the linking elements 101.13 may also be undulated elements which are undulated along the longitudinal direction LD. With certain variants, the linking elements 101.13 may be either of a filament element, a surgical suture filament, a tongue element protruding from the sleeve unit 101.2, and a wire element. Either of these configurations is particularly beneficial in that the design and flexibility of the linking elements supports adaptation of the sleeve unit to the vessel topography. Either of these configurations is particularly beneficial in that the design and flexibility of the linking elements 101.13 supports adaptation of the sleeve unit 101.2 to the vessel topography.

With certain further variants (not shown), the linking means 101.12 may comprise a perforated linking sleeve section, the linking sleeve section having a degree of perforation which is at least 85%, preferably at least 90%, more preferably 86% to 94%, in particular, 89% to 92%. The perforated linking sleeve section may be formed monolithically with at least one of the sleeve unit 101.2 and the anchoring section 101.1. Here, a particularly simple to manufacture and robust configuration may be achieved.

It will be appreciated that the sleeve unit 101.2 may have any desired and suitable configuration yielding a thin-walled and highly flexible component which snugly conforms to the vessel topography. Preferably, the sleeve unit 101.2 has a wall thickness ranging from 0.05 mm to 2.0 mm, preferably 0.1 mm to 1.0 mm, more preferably 0.25 mm to 0.5 mm. Any desired and suitable materials may be used for the sleeve unit 101.2. With certain variants, the sleeve unit 101.2 is made from a material selected from a material group consisting of a polymer material, polyethylene (PE), polytetrafluoroethylene (PTFE), polyurethane (PU), a bioresorbable material, a lactide caprolactone, allogeneic pericardium, matrix based and/or tissue engineered material, and combinations thereof.

It will be appreciated that the anchoring and support functionality as described herein may be achieved in any desired and suitable way ensuring proper contact forces at the aorta 104. In the present example, to this end, the anchoring unit 101.1 and possibly the support unit 106 supporting the sleeve unit 101.2 at the downstream sleeve unit end 101.8 comprise a collapsible and a self-expanding structure 101.6, 106. With certain preferred variants, the collapsible and expandable structure 101.6, 106 is made from a material selected from a material group consisting of a shape memory material, a metal, a nickel titanium alloy, a polymer material, a bioresorbable material, a magnesium based material, and combinations thereof. With certain variants, the collapsible and expandable structure 101.6, 106 may be a grid or a wire structure, such as the stent element 101.6, 106 shown in FIGS. 1 and 2. Any of these variants yields particularly beneficial designs with proper and stable application of contact forces.

The catheter device 102 has a proximal end 102.1 (located external to the patient's body and manipulated by the surgeon or cardiologist) and a distal end 101.2 (located in the aorta 104 during implantation). As can be seen from FIGS. 3 and 4 (showing the distal end 102.2 of the catheter device 102), the catheter device 102, in a catheter insertion state, receives the endograft device 101 within an endograft receptacle 102.3 formed at the distal end 102.2 of the catheter device 102. The endograft device 101 is in a collapsed state when it is received in the endograft receptacle 102.3 (see FIG. 3). The catheter device is configured to release, in an endograft release state, the endograft device 101 from the endograft receptacle 102.3.

It will be appreciated that, radio-opaque markers (not shown) are integrated at certain suitable locations of the endograft device 101, e.g., at the upstream downstream ends of both, the stent element 101.6 and the sleeve unit 101.2, as well as at various additional segments of the entire endograft device 101. This enables quick and simple verification of the proper placement of the endograft device 101 in the aorta 104 using suitable imaging techniques. In addition or as an alternative, such radio-opaque markers may also be integrated in the catheter device 102.

In the present example, the catheter device 102 comprises an inner catheter core 102.4 and an outer catheter sleeve 102.5, wherein the endograft receptacle 102.3 is formed between the inner catheter core 102.4 and the outer catheter sleeve 102.5 when the outer catheter sleeve 102.5 is in a distally advanced state (see FIG. 3). The outer catheter sleeve 102.5 is proximally retractable with respect to the inner catheter core 102.4 to release the endograft device 101 (see FIG. 4).

As, in the present example, the anchoring unit 101.1 and the possibly present support unit 106 at the downstream sleeve unit end 101.8 comprise a self-expanding structure expanding upon release from the outer catheter sleeve 102.5, particularly simple release may be achieved. With certain variants, the inner catheter core 102.4 may also be configured to at least support radial expansion of the sleeve unit 101.2. This may happen when retracting the inner catheter core 102.4, in the endograft release state, proximally from the endograft device 101 as will be explained below. By this means, particularly good adaptation and snug fit of the sleeve unit 101.2 to the blood vessel topography may be achieved.

As can be seen from FIG. 4, support of the radial expansion of the sleeve unit 101.2 is achieved by means of a catheter core expansion section 102.6 which is configured to radially expand to support radial expansion of the sleeve unit 101.2. In the present example, the catheter core expansion section 102.6 is configured to radially expand when the outer catheter sleeve 102.5 is proximally retracted with respect to the inner catheter core 102.4 (see FIG. 4).

Expansion of the catheter core expansion section 102.6 may be achieved in any suitable way. For example, the catheter core expansion section 102.6 may be made from an expandable material forming one or more suitable chambers to be filled with a suitable fluid to provide expansion (similar to known balloon catheter devices, for example). In the present example, a particularly simple yet robust and efficient solution is achieved in that the inner catheter core 102.4 comprises a plurality of elastic arms 102.7 which radially expand and engage the sleeve unit 101.2 to support radial expansion of the sleeve unit 101.2 when the outer catheter sleeve 102.5 is proximally retracted with respect to the inner catheter core 102.4 (see FIG. 4). This configuration has the great advantage that the elastic arms 102.7 may be configured to only marginally block blood flow within the aorta 104 which further supports expansion of the sleeve unit 101.2.

With certain variants, the elastic arms 102.7 are distributed, in particular, at least substantially evenly distributed, along a circumference of the inner catheter core 102.4. In the present example, the elastic arms 102.7 are retracted proximally with respect to the sleeve unit 101.2 and to slide along the sleeve unit 101.2 when being retracted proximally. This achieves a particularly simple configuration with proper contact between the sleeve unit 101.2 and the aorta 104 along the length of the sleeve unit 101.2. As can be seen from FIG. 4, the elastic arms 102.7 have a further radially extended relaxed state (going radially beyond the fully expanded anchoring unit 101.1) as indicated by the dashed contour 107. This ensures application of proper expansion forces to the sleeve unit 101.2 and particularly good adaptation and snug fit of the sleeve unit 101.2 to almost arbitrarily complex blood vessel topographies, such as is the case at the location where the left subclavian artery (or subclavia sinistra) 104.5 branches off from the aortic arch 104.2 as it is schematically indicated by the dashed contour representing the aorta 104 in FIG. 4.

In the present example, the elastic arms 102.7 are retracted into the outer catheter sleeve 102.5 after having been proximally retracted from the sleeve unit 101.2. This enables simple and safe removal of the catheter device 102 after release of the endograft device 101.

Preferably, the inner catheter core 102.4 and the outer catheter sleeve 102.5 are configured such that a proximal retraction motion of the elastic arms 102.7 and a proximal retraction motion of the outer catheter sleeve 102.5 are synchronized such that the proximal retraction motion of the elastic arms 102.7 follows the proximal retraction motion of the outer catheter sleeve 102.5 with a predefined delay. For example, retraction of the outer catheter sleeve 102.5 relative to inner catheter core 102.4 may be limited to the situation as shown in FIG. 4. Once this state is reached, the relative position between the outer catheter sleeve 102.5 and the inner catheter core 102.4 is locked. Further retraction of the outer catheter sleeve 102.5 then goes along with a simultaneous retraction motion of the inner catheter core 102.4 from the endograft device 101. This enables simple and safe release of the endograft device 101 from the catheter device 102.

It will be appreciated that, while the catheter device 102 in FIGS. 3 and 4, for the sake of simplicity is shown in a straight configuration, the catheter device 102 can be manipulated or steered into any desired and suitable curved configuration to at least largely match the geometry of the blood vessel, e.g., the aorta 104 and the aortic arch 104.2, respectively. Such catheter steering is well known in the art and will therefore not be addressed in greater detail here.

It will be further appreciated that, while the endograft device 101 has only been described with reference to configurations with flared end sections of the sleeve unit 101.2, simple entirely cylindrical configurations may also be used as is indicated by the dashed contour 108 in FIG. 2.

It will be further appreciated that, with the present invention, it is possible to achieve, in a simple and efficient manner, improved treatment of ruptures 104.9 in blood vessels, such as the aorta 104, at locations with complex vessel geometry. This is due to the fact that the anchoring unit 101.1, in the implanted state of the endograft device 101, is located upstream of the thin and flexible sleeve unit 101.2 such that at least the upstream part of the sleeve unit 101.2 sealingly covering the rupture 104.9 is unsupported along its circumference. This longitudinally non-overlapping arrangement of the flexible sleeve unit 101.2 and the anchoring unit 101.1 frees considerable adaptation capacity of the expanding sleeve unit 101.2, especially in the radial direction of the endograft device 101 or blood vessel 104, respectively. As noted, this is due to the fact that constraints in the freedom of the sleeve unit 101.2 to adapt to the vessel topography which conventional designs impose by a (longitudinally) overlapping or embedded anchoring unit are largely removed. This allows the unsupported sleeve unit 101.2 to at least largely freely and closely conform (under the supporting influence of the blood pressure inside the blood vessel 104) to the vessel topography around the rupture 104.9, and, therefore, tightly and reliably seal the rupture 104.9.

While the present invention, in the foregoing has been mainly described in the context of treatment of aneurysms in the aorta 104 at the aortic arch, it will be appreciated that the invention may also be used in any other configuration where ruptures in body vessels have to be treated.

1. An endograft device for treatment of ruptures in one or more inner layers of a blood vessel, in particular, an aorta, comprising
an anchoring unit and
a sleeve unit,
wherein
said endograft device defines an upstream endograft end and a downstream endograft end, upstream and downstream being defined, in an implanted state of the endograft device,in relation to a general natural blood flow defining a blood flow direction within said blood vessel;
said anchoring unit is a collapsible unit located at said upstream endograft end;
said anchoring unit is configured to anchor, in said implanted state, said endograft device within said blood vessel by engaging an inner surface of said blood vessel with an expanded anchoring section;
said sleeve unit is located downstream of said anchoring unit;
said sleeve unit is a thin-walled foldable element defining a longitudinal direction, a radial direction, a circumferential direction, an upstream sleeve unit end and a upstream sleeve unit end;
characterized in that
said sleeve unit comprises an unsupported section located at said upstream sleeve unit end,
said unsupported section extends along said longitudinal direction and, at least in said implanted state, is unsupported over its entire circumference,
said sleeve unit is configured to be expanded, in said implanted state, to rest against an inner wall of said blood vessel to sealingly cover said rupture with said unsupported section.

2. The endograft device according to claim 1, wherein at least one of the following applies:
said unsupported section, along said longitudinal direction, extends over at least 10%, preferably at least 20%, more preferably 25% to 100%, in particular, 40% to 80%, of said sleeve unit; and
said unsupported section, along said longitudinal direction, extends over 10 mm to 100 mm, preferably 20 mm to 80 mm, more preferably 40 mm to 60 mm.

3. The endograft device according to claim 1, wherein
said sleeve unit comprises at least one supported section,
said at least one supported section is located adjacent to said unsupported section and at a distance from said upstream sleeve unit end,
said at least one supported section, in said implanted state, is supported over its circumference, by at least one expandable support unit,
wherein, in particular, at least one of the following applies:
said at least one supported section is located at said upstream sleeve unit end;
said at least one supported section, along said longitudinal direction, extends over at least 10%, preferably at least 15%, more preferably 20% to 60%, in particular, 30% to 50%, of said sleeve unit;
said at least one supported section, along said longitudinal direction, extends over 10 mm to 60 mm, preferably 15 mm to 50 mm, more preferably 20 mm to 40 mm;
said least one supported section, in said radial direction, is supported by said at least one support unit on and outer side and/or on an inner side of said sleeve unit.

4. The endograft device according to claim 1, wherein
said anchoring section is configured to be expanded, in said implanted state, to a maximum anchoring unit diameter, a circle having said maximum anchoring unit diameter defining a reference circumferential length of said anchoring unit;
said sleeve unit, in a relaxed and fully expanded state free of stress in said circumferential direction, at said upstream sleeve unit end, has a relaxed expanded circumferential length;
said anchoring unit and said sleeve unit are arranged and configured such that, in said implanted state, said relaxed expanded circumferential length of said upstream sleeve unit end is larger than said reference circumferential length of said anchoring unit.

5. The endograft device according to claim 4, wherein at least one of the following applies:
said anchoring unit and said sleeve unit are arranged and configured such that, in said implanted state, said relaxed expanded circumferential length of said upstream sleeve unit end is at least 101%, preferably at least 105%, more preferably 110% to 130%, in particular, 115% to 125%, of said reference circumferential length of said anchoring unit;
said relaxed expanded circumferential length of said upstream sleeve unit end is 55 mm to 75 mm, preferably 60 mm to 70 mm, more preferably 62 mm to 65 mm;
said reference circumferential length of said anchoring unit is 50 mm to 70 mm, preferably 55 mm to 65 mm, more preferably 58 mm to 62 mm.

6. The endograft device according to claim 1, wherein
said sleeve unit is configured to have, in a relaxed and fully expanded state free of stress in said circumferential direction, a shape that is at least one of (i) at least section-wise at least substantially cylindrical and (ii) flared, along said longitudinal direction, towards at least one of said upstream sleeve unit end and said upstream sleeve unit end;
wherein, in particular, at least one of the following applies:
said sleeve unit, in said relaxed and fully expanded state, along said longitudinal direction, has a sleeve unit minimum diameter and is flared towards said upstream sleeve unit end to have an upstream flared diameter at said upstream sleeve unit end, said upstream flared diameter being at least one of (i) 101%, preferably at least 103%, more preferably 105% to 130%, in particular, 110% to 125%, of said sleeve unit minimum diameter, and (ii) at least 18 mm to 25 mm, preferably 19 mm to 23 mm, more preferably 20 mm to 22 mm;
said sleeve unit, in said relaxed and fully expanded state, along said longitudinal direction, has a sleeve unit minimum diameter and is flared towards said upstream sleeve unit end to have a downstream flared diameter at said upstream sleeve unit end, said downstream flared diameter being at least one of (i) 101%, preferably at least 103%, more preferably 105% to 130%, in particular, 110% to 125%, of said sleeve unit minimum diameter, and (ii) at least 18 mm to 25 mm, preferably 19 mm to 23 mm, more preferably 20 mm to 22 mm;
said anchoring section, in said implanted state, has a maximum anchoring unit diameter and said sleeve unit, in said relaxed and fully expanded state, along said longitudinal direction, has a sleeve unit minimum diameter, said sleeve unit minimum diameter being at least one of (i) 101%, preferably at least 103%, more preferably 105% to 130%, in particular, 110% to 125%, of said maximum anchoring unit diameter, and (ii) at least 16 mm to 22 mm, preferably 17 mm to 21 mm, more preferably 18 mm to 20 mm;
said sleeve unit, to provide said flared shape in said relaxed and fully expanded state, has a collapsed state where at least one folded section is formed within said sleeve unit.

7. The endograft device according to claim 1, wherein
said sleeve unit is spaced, in said longitudinal direction, from said anchoring unit;
wherein, in particular, at least one of the following applies:
said anchoring section is configured to be expanded, in said implanted state, to a maximum anchoring unit diameter, and said sleeve unit, in said longitudinal direction, is located at a distance D from said anchoring unit, said distance D being at least 5%, preferably at least 10%, more preferably 15% to 100%, in particular, 25% to 75%, of said maximum anchoring unit diameter;
said sleeve unit, in said longitudinal direction, is located at a distance D from said anchoring unit, said distance D being at least 1 mm to 20 mm, preferably 3 mm to 15 mm, more preferably 5 mm to 10 mm.

8. The endograft device according to claim 1, wherein
said sleeve unit is linked, in said longitudinal direction, to said anchoring unit by linking means of said endograft device, in particular, by a linking section of said endograft device;
wherein, in particular, at least one of the following applies:
said linking means comprises a plurality of linking elements mutually spaced and distributed, in particular, evenly distributed, along said circumferential direction;
said linking means comprises a plurality of linking elements, at least one of said linking elements being a slender element elongated along said longitudinal direction;
said linking means comprises a plurality of linking elements, at least one of said linking elements being an undulated element undulated along said longitudinal direction;
said linking means comprises a plurality of linking elements, at least one of said linking elements being a flexible element exhibiting a high flexibility in a direction transverse to said longitudinal direction;
said linking means comprises a plurality of linking elements, at least one of said linking elements being one of a filament element, a surgical suture filament, a tongue element protruding from said sleeve unit, and a wire element;
said linking means comprises a perforated linking sleeve section, said linking sleeve section having a degree of perforation which is at least 85%, preferably at least 90%, more preferably 86% to 94%, in particular, 89% to 92%, said perforated linking sleeve section, in particular, being formed monolithically with at least one of said sleeve unit and said anchoring section.

9. The endograft device according to claim 1, wherein at least one of the following applies:
said sleeve unit has a wall thickness ranging from 0.05 mm to 2.0 mm, preferably 0.1 mm to 1.0 mm, more preferably 0.25 mm to 0.5 mm.; and
said sleeve unit is made from a material selected from a material group consisting of a polymer material, polyethylene (PE), polytetrafluoroethylene (PTFE), polyurethane (PU), a bioresorbable material, a lactide caprolactone, allogeneic pericardium, matrix based and/or tissue engineered material, and combinations thereof.

10. The endograft device according to claim 1, wherein at least one of the following applies:
said anchoring unit and/or a support unit supporting said sleeve unit at said upstream sleeve unit end comprises a collapsible and expandable structure, in particular, a self-expanding structure;
wherein, in particular, at least one of the following applies:
said collapsible and expandable structure is made from a material selected from a material group consisting of a shape memory material, a metal, a nickel titanium alloy, a polymer material, a bioresorbable material, a magnesium based material, and combinations thereof;
said collapsible and expandable structure comprises ate least one of a grid and a wire structure;
said collapsible and expandable structure comprises at least one stent element.

11. A kit for treatment of ruptures in one or more inner layers of a blood vessel, in particular, an aorta, comprising
at least one endograft device according to claim 1, and
a catheter device, in particular, for percutaneous insertion into a blood vessel of a patient;
wherein
said catheter device has a proximal end and a distal end,
said catheter device is configured to receive, in a catheter insertion state, said endograft device within an endograft receptacle formed at said distal end of said catheter device, said endograft device being in a collapsed state when received in said endograft receptacle, and
said catheter device is configured to release, in an endograft release state, said endograft device from said endograft receptacle.

12. The kit according to claim 11, wherein said catheter device comprises an inner catheter core and an outer catheter sleeve;
said endograft receptacle being formed between said inner catheter core and said outer catheter sleeve when said outer catheter sleeve is in a distally advanced state;
said outer catheter sleeve is proximally retractable with respect to said inner catheter core to release said endograft device
wherein, in particular, at least one of the following applies:
said anchoring unit and/or a support unit of said endograft device supporting said sleeve unit at said upstream sleeve unit end comprises a self-expanding structure expanding upon release from said outer catheter sleeve,
said inner catheter core is configured to at least support radial expansion of said sleeve unit, in particular, when retracting said inner catheter core, in said endograft release state, proximally from said endograft device.

13. A catheter device for inserting an endograft device according to claim 1 into a blood vessel, in particular, an aorta, for
treatment of ruptures in one or more inner layers of said blood vessel, said
catheter device comprising
an inner catheter core and
an outer catheter sleeve;
wherein
an endograft receptacle is formed between said inner catheter core and said outer catheter sleeve for receiving said endograft device when said outer catheter sleeve is in a distally advanced state;
said outer catheter sleeve is proximally retractable with respect to said inner catheter core to release said endograft device from said endograft receptacle in an endograft release state;
said inner catheter core is configured to at least support radial expansion of said sleeve unit, in particular, when retracting said inner catheter core, in said endograft release state, proximally from said endograft device.

14. The catheter device according to claim 13, wherein at least one of the following applies:
said inner catheter core comprises a catheter core expansion section which is configured to radially expand to at least support radial expansion of said sleeve unit,
said inner catheter core comprises a catheter core expansion section which is configured to radially expand when said outer catheter sleeve is proximally retracted with respect to said inner catheter core;

15. The catheter device according to claim 14, wherein
said inner catheter core comprises a plurality of elastic arms, said elastic arms being configured to radially expand and engage said sleeve unit to support radial expansion of said sleeve unit when said outer catheter sleeve is proximally retracted with respect to said inner catheter core;
wherein, in particular, at least one of the following applies: said elastic arms are distributed, in particular, at least substantially evenly distributed, along a circumference of said inner catheter core;
said elastic arms are configured be retracted proximally with respect to said sleeve unit and to slide along said sleeve unit when being retracted proximally;
said elastic arms are configured be retracted into said outer catheter sleeve after having been proximally retracted from said sleeve unit;
said inner catheter core and said outer catheter sleeve are configured such that a proximal retraction motion of said elastic arms and a proximal retraction motion of said outer catheter sleeve are synchronized such that said proximal retraction motion of said elastic arms follows said proximal retraction motion of said outer catheter sleeve with a predefined delay.
